(19) United States
(12) Patent Application Publication
(10) Pub. No.: US 2019/0209834 A1
(43) Pub. Date: Jul. 11, 2019

(54) IMPLANTABLE STIMULATION LEADS FOR GLIAL MODULATION AND METHODS OF MAKING AND USING SAME

(71) Applicant: Boston Scientific Neuromodulation Corporation, Valencia, CA (US)

(72) Inventors: Tianhe Zhang, Studio City, CA (US); Bradley Lawrence Hershey, Carrollton, TX (US); Rosana Esteller, Santa Clarita, CA (US)

(21) Appl. No.: 16/242,370

(22) Filed: Jan. 8, 2019

Related U.S. Application Data
(60) Provisional application No. 62/616,362, filed on Jan. 11, 2018.

(51) Int. Cl. A61N 1/05; A61N 1/36

(52) U.S. Cl. CPC A61N 1/0553; A61N 1/36062; A61N 1/0534; A61N 1/36021; A61N 1/0556

(57) ABSTRACT

Paddle or percutaneous leads for gliomodulation include electrodes arranged for preferentially stimulating glial cells. The leads may also include at least one non-electrical sensor or optical stimulator.

### CROSS-REFERENCE TO RELATED APPLICATIONS

This application claims the benefit under 35 U.S.C. § 119(e) of U.S. Provisional Patent Application Ser. No. 62/616,362, filed Jan. 11, 2018, which is incorporated herein by reference.

### FIELD

The present invention is directed to the area of implantable electrical stimulation leads and methods of making and using the leads, as well as systems containing the leads. The present invention is also directed to leads for glial stimulation including stimulation of glial cells in the spinal cord.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a paddle lead that includes a paddle body; a first column of electrodes disposed along the paddle body; a second column of electrodes disposed along the paddle body; at least one of a non-electrical sensor or an optical stimulator disposed along the paddle body and between the first and second columns; at least one lead body extending from the paddle body; and terminals disposed along the at least one lead body and electrically coupled to the electrodes of the first and second columns.

In at least some embodiments, the at least one of the non-electrical sensor or the optical stimulator includes a plurality of the non-electrical sensors arranged in a medial column between the first and second columns. In at least some embodiments, the at least one of the non-electrical sensor or the optical stimulator includes a plurality of the optical stimulators arranged in a medial column between the first and second columns. In at least some embodiments, the at least one of the non-electrical sensor or the optical stimulator includes a plurality of the non-electrical sensors and optical stimulators arranged in a medial column between the first and second columns. In at least some embodiments, the at least one of the non-electrical sensor or the optical stimulator includes at least one of the non-electrical sensors selected from an optical sensor, a piezoelectric sensor, a chemical sensor, or an accelerometer. In at least some embodiments, the at least one of the non-electrical sensor or the optical stimulator includes at least one of the optical stimulators selected from a light emitting diode, an organic light emitting diode, or an optical fiber coupleable to a light source.

In at least some embodiments, at least one of the electrodes has a dimension of at least 4 mm. In at least some embodiments, the first column of electrodes includes no more than eight of the electrodes and the first column has al dimension of at least 120 mm from a distal end of a distal-most one of the electrodes to a proximal end of the proximal-most one of the electrodes. In at least some embodiments, the first and second columns are separated by a center-to-center distance of at least 7 mm. In at least some embodiments, the first and second columns are configured and arranged to modulate glial cells preferentially over neurons.

Another embodiment is a percutaneous lead that includes a lead body having a distal portion and a proximal portion; electrodes disposed along the distal portion of the lead body; at least one non-electrical sensor disposed along the distal portion of the lead body; at least one optical stimulator disposed along the distal portion of the lead body; and terminals disposed along the proximal portion of the lead body and electrically coupled to the electrodes.

In at least some embodiments, the at least one non-electrical sensor comprises at least one of an optical sensor, a piezoelectric sensor, a chemical sensor, or an accelerometer. In at least some embodiments, the at least one optical stimulator comprises at least one of a light emitting diode, an organic light emitting diode, or an optical fiber coupleable to a light source.

In at least some embodiments, at least one of the electrodes has a dimension of at least 4 mm. In at least some embodiments, the plurality of electrodes includes no more than eight of the electrodes with a dimension of at least 120 mm from a distal end of a distal-most one of the electrodes to a proximal end of the proximal-most one of the electrodes. In at least some embodiments, the electrodes are configured and arranged to modulate glial cells preferentially over neurons.

Yet another embodiment is an electrical stimulating system that includes any of the leads described above; and a control module coupleable to the percutaneous lead. The control module includes a housing, and an electronic subassembly disposed in the housing.

A further embodiment is a method for glial modulation that includes implanting any of the leads describe above adjacent a spinal cord of a patient; and delivering electrical stimulation through one or more of the electrodes of the paddle lead to modulate glial cells in the spinal cord.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle body coupled to a control module via lead bodies, according to the invention;

FIG. 2 is a schematic view of another embodiment of an electrical stimulation system that includes a percutaneous lead body coupled to a control module via a lead body, according to the invention;

FIG. 3A is a schematic view of one embodiment of a plurality of connector assemblies disposed in the control module of FIG. 1, the connector assemblies configured to receive the proximal portions of the lead bodies of FIG. 1, according to the invention;

FIG. 3B is a schematic view of one embodiment of a connector assembly disposed in the control module of FIG. 2, the connector assembly configured to receive the proximal portion of one of the lead body of FIG. 2, according to the invention;

FIG. 3C is a schematic view of one embodiment of a proximal portion of the lead body of FIG. 2, a lead extension, and the control module of FIG. 2, the lead extension configured to couple the lead body to the control module, according to the invention;

FIG. 4 is a schematic cross-sectional view of a portion of the spinal cord;

FIG. 5 is a schematic, side view of one embodiment of a portion of a paddle lead with spaced apart columns of electrodes for glial modulation, according to the invention;

FIG. 6A is a schematic, side view of one embodiment of a portion of a percutaneous lead including at least one non-electrical sensor or optical stimulator, according to the invention;

FIG. 6B is a schematic, side view of another embodiment of a portion of a percutaneous lead including at least one non-electrical sensor or optical stimulator, according to the invention;

FIG. 6C is a schematic, side view of one embodiment of an arrangement of percutaneous leads, according to the invention;

FIG. 6D is a schematic, side view of another embodiment of an arrangement of percutaneous leads, according to the invention;

FIG. 6E is a schematic, side view of a third embodiment of an arrangement of percutaneous leads, according to the invention; and

FIG. 7 is a schematic overview of one embodiment of components of a stimulation system, including an electronic subassembly disposed within a control module, according to the invention.

### DETAILED DESCRIPTION

The present invention is directed to the area of implantable electrical stimulation leads and methods of making and using the leads, as well as systems containing the leads. The present invention is also directed to leads for glial stimulation including stimulation of glial cells in the spinal cord.

The present patent application incorporates by reference, in its entirety, U.S. Provisional Patent Application No. 62/616,360 entitled “Methods and Systems for Stimulation for Glial Modulation”, filed on even date herewith (Attorney Docket No. BSNC-1-686.0).

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 6,175,710; 6,224,450; 6,271,094; 6,295,944; 6,364,278; 6,391,985; 8,473,061; 8,571,665; and 8,792,993; and U.S. patent Applications Publication Nos. 2007/0150036; 2009/0187222; 2009/0276021; 2010/0076535; 2010/0268298; 2011/0004267; 2011/0005069; 2011/0078900; 2011/0130816; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2013/0197424; 2013/0197602; 2014/0039587; 2014/0353001; 2014/0358207; 2014/0358208; 2014/0358209; 2014/0358210; 2015/0018915; 2015/0045864; 2015/0051681; 2015/0066120; 2015/0151113; and 2016/0228692, all of which are incorporated by reference in their entireties.

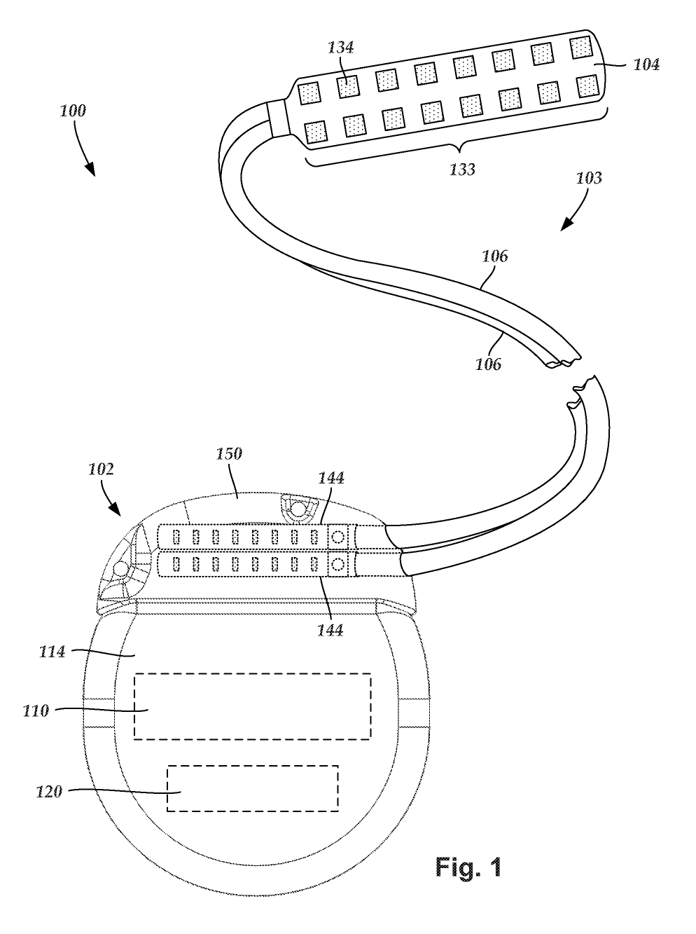

FIG. 1 illustrates schematically one embodiment of an electrical stimulation system 100. The electrical stimulation system includes a control module (e.g., a stimulator or pulse generator) 102 and a lead 103. The lead 103 includes a paddle body 104 and one or more lead bodies 106 coupling the control module 102 to the paddle body 104. The paddle body 104 and the one or more lead bodies 106 form the lead 103. The paddle body 104 typically includes a plurality of electrodes 134 that form an array of electrodes 133. The control module 102 typically includes an electronic subassembly 110 and an optional power source 120 disposed in a sealed housing 114. In FIG. 1, two lead bodies 106 are shown coupled to the control module 102.

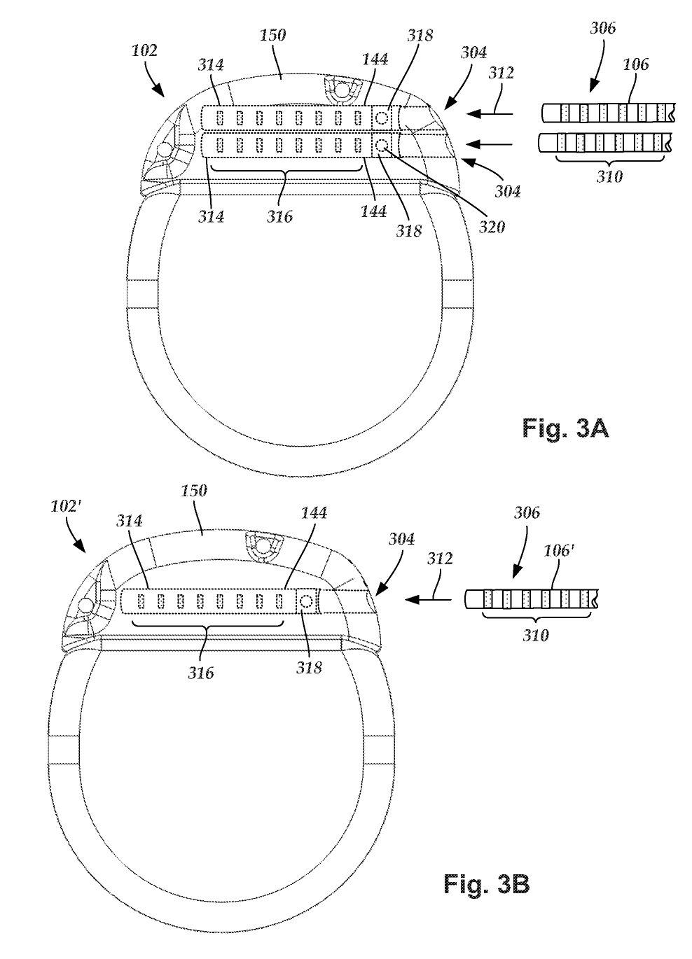

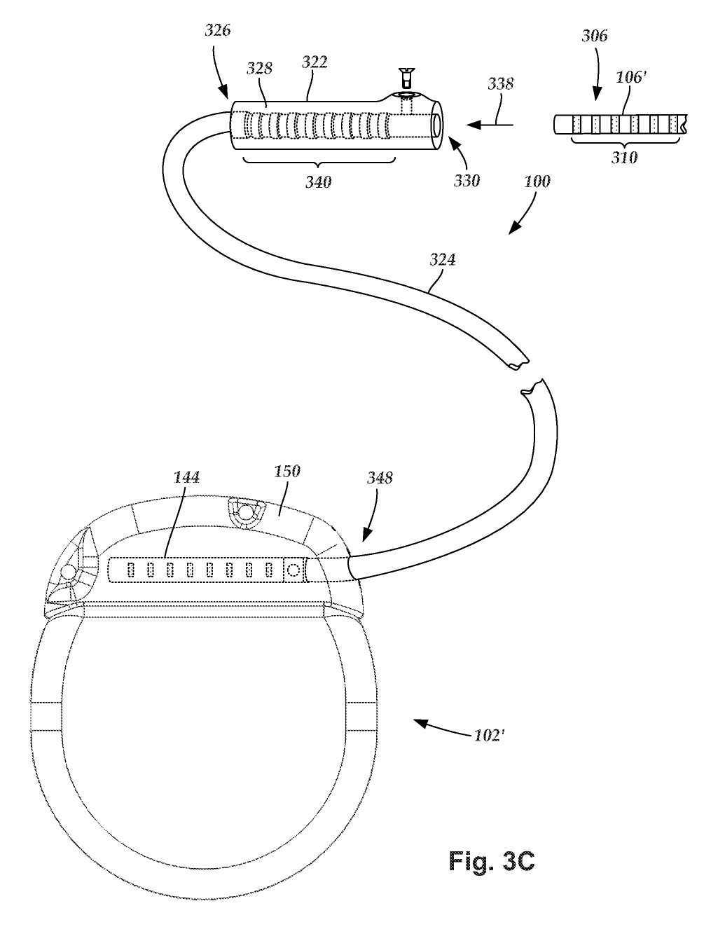

The control module 102 typically includes one or more connector assemblies 144 into which the proximal end of the one or more lead bodies 106 can be plugged to make an electrical connection via connector contacts (e.g., 316 in FIG. 3A) disposed in the connector assembly 144 and terminals (e.g., 310 in FIG. 3A) on each of the one or more lead bodies 106. The connector contacts are coupled to the electronic subassembly 110 and the terminals are coupled to the electrodes 134. In FIG. 1, two connector assemblies 144 are shown.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 324 (see FIG. 3C) can be disposed between the one or more lead bodies 106 and the control module 102 to extend the distance between the one or more lead bodies 106 and the control module 102.

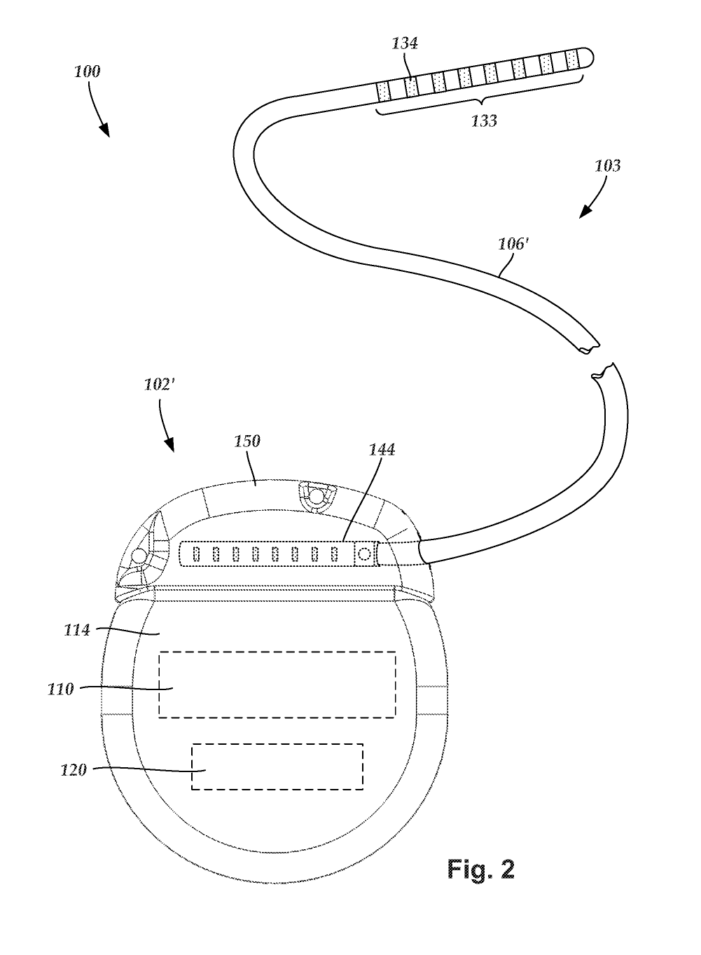

It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of a lead body 106′ forming a percutaneous lead 103, as illustrated in FIG. 2. The percutaneous lead may be isodiametric along the length of the lead body 106″. The lead body 106′ can be coupled with a control module 102′ with a single connector assembly 144.

The electrical stimulation system or components of the electrical stimulation system, including one or more of the lead bodies 106, the control module 102, and, in the case of a paddle lead, the paddle body 104, are typically implanted into the body of a patient. The electrical stimulation system can be used for a variety of applications including, but not limited to, spinal cord stimulation, brain stimulation, neural stimulation, glial modulation, muscle activation via stimulation of nerves innervating muscle, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, titanium, or rhenium.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead 103 to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIG. 3A) are typically disposed at the proximal end of the one or more lead bodies 106 for connection to corresponding conductive contacts (e.g., 316 in FIG. 3A) in connector assemblies (e.g., 144 in FIG. 1) disposed on, for example, the control module 102 (or to other devices, such as conductive contacts on a lead extension, an operating room cable, a splitter, an adaptor, or the like).

Conductive wires (not shown) extend from the terminals (e.g., 310 in FIG. 3A) to the electrodes 134. Typically, one or more electrodes 134 are electrically coupled to a terminal (e.g., 310 in FIG. 3A). In some embodiments, each terminal (e.g., 310 in FIG. 3A) is only coupled to one electrode 134.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, each of the two lead bodies 106 includes eight terminals that are shown coupled with eight conductive contacts disposed in a different one of two different connector assemblies 144.

FIG. 3A is a schematic side view of one embodiment of a plurality of connector assemblies 144 disposed on the control module 102. In at least some embodiments, the control module 102 includes two connector assemblies 144. In at least some embodiments, the control module 102 includes four connector assemblies 144. In FIG. 3A, proximal ends 306 of the plurality of lead bodies 106 are shown configured for insertion to the control module 102. FIG. 3B is a schematic side view of one embodiment of a single connector assembly 144 disposed on the control module 102′. In FIG. 3B, the proximal end 306 of the single lead body 106′ is shown configured for insertion to the control module 102′.

In FIGS. 3A and 3B, the one or more connector assemblies 144 are disposed in the header 150. In at least some embodiments, the header 150 defines one or more ports 304 into which the proximal end(s) 306 of the one or more lead bodies 106/106′ with terminals 310 can be inserted, as shown by directional arrows 312, in order to gain access to the connector contacts disposed in the one or more connector assemblies 144.

The one or more connector assemblies 144 each include a connector housing 314 and a plurality of connector contacts 316 disposed therein. Typically, the connector housing 314 defines a port (not shown) that provides access to the plurality of connector contacts 316. In at least some embodiments, one or more of the connector assemblies 144 further includes a retaining element 318 configured to fasten the corresponding lead body 106/106′ to the connector assembly 144 when the lead body 106/106′ is inserted into the connector assembly 144 to prevent undesired detachment of the lead body 106/106′ from the connector assembly 144. For example, the retaining element 318 may include an aperture 320 through which a fastener (e.g., a set screw, pin, or the like) may be inserted and secured against an inserted lead body 106/106′.

When the one or more lead bodies 106/106′ are inserted into the one or more ports 304, the connector contacts 316 can be aligned with the terminals 310 disposed on the one or more lead bodies 106/106′ to electrically couple the control module 102 to the electrodes (134 of FIG. 1) disposed at a distal end of the one or more lead bodies 106. Examples of connector assemblies in control modules are found in, for example, U.S. Pat. Nos. 7,244,150 and 8,224,450, which are incorporated by reference in their entireties.

In at least some embodiments, the electrical stimulation system includes one or more lead extensions. The one or more lead bodies 106/106′ can be coupled to one or more lead extensions which, in turn, are coupled to the control module 102/102′. In FIG. 3C, a lead extension connector assembly 322 is disposed on a lead extension 324. The lead extension connector assembly 322 is shown disposed at a distal end 326 of the lead extension 324. The lead extension connector assembly 322 includes a contact housing 328. The contact housing 328 defines at least one port 330 into which a proximal end 306 of the lead body 106′ with terminals 310 can be inserted, as shown by directional arrow 338. The lead extension connector assembly 322 also includes a plurality of connector contacts 340. When the lead body 106′ is inserted into the port 330, the connector contacts 340 disposed in the contact housing 328 can be aligned with the terminals 310 on the lead body 106 to electrically couple the lead extension 324 to the electrodes (134 of FIG. 1) disposed at a distal end (not shown) of the lead body 106′.

The proximal end of a lead extension can be similarly configured as a proximal end of a lead body. The lead extension 324 may include a plurality of conductive wires (not shown) that electrically couple the connector contacts 340 to terminal on a proximal end 348 of the lead extension 324. The conductive wires disposed in the lead extension 324 can be electrically coupled to a plurality of terminals (not shown) disposed on the proximal end 348 of the lead extension 324. In at least some embodiments, the proximal end 348 of the lead extension 324 is configured for insertion into a lead extension connector assembly disposed in another lead extension. In other embodiments (as shown in FIG. 3C), the proximal end 348 of the lead extension 324 is configured for insertion into the connector assembly 144 disposed on the control module 102′.

It will be understood that the control modules 102/102′ can receive either lead bodies 106/106′ or lead extensions 324. It will also be understood that the electrical stimulation system 100 can include a plurality of lead extensions 224. For example, each of the lead bodies 106 shown in FIGS. 1 and 3A can, alternatively, be coupled to a different lead extension 224 which, in turn, are each coupled to different ports of a two-port control module, such as the control module 102 of FIGS. 1 and 3A.

Stimulation of patient tissue, such as the spinal cord, can be useful in reducing pain and providing other therapy. There is an increasing interest in the role of glial cells in chronic pain. Conventional spinal cord stimulation, however, is generally focused solely on modulating neuronal cells. Electrode configurations and stimulation patterns, including migratory stimulation, can be used to target glial cells, such as microglia, astrocytes, or oligodendrocytes or the like or any combination thereof, to promote healing, reduce inflammation, or relieve pain (or any combination thereof) In at least some embodiments, mechanisms such as, for example, electrotaxis, chemotaxis, galvanotaxis, or electromechanical effects produced by Lorenz interactions caused by an applied waveform (or any combination thereof) can be utilized to modulate glial cells.

In at least some embodiments, the glial cells that are to be stimulated reside in the dorsal horns. In contrast, conventional paddle leads for spinal cord stimulation typically have electrodes arranged to stimulate the neurons in the spinal columns.

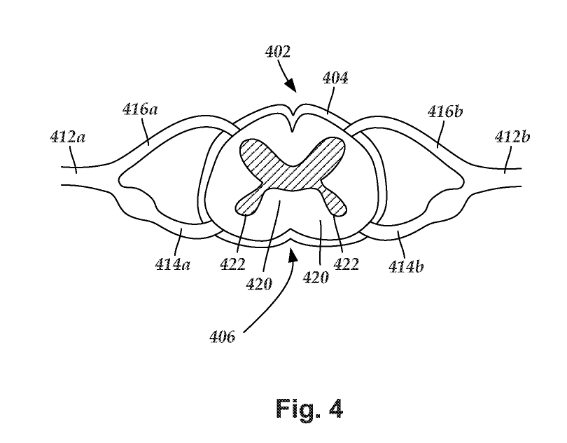

FIG. 4 schematically illustrates a transverse cross-sectional view of a spinal cord 402 surrounded by dura 404. The spinal cord 402 includes the dorsal (or posterior) column 420 and the dorsal (or posterior) horns 422. The spinal cord 402 also includes a midline 406 and multiple levels from which spinal nerves 412a and 412b extend. In FIG. 4, the spinal nerves 412a and 412b are shown attaching to the spinal cord 402 at a particular spinal cord level via corresponding dorsal roots 414a and 414b and ventral (or anterior) roots 416a and 416b. Typically, the dorsal roots 414a and 414b relay sensory information into the spinal cord 402 and the ventral roots 416a and 416b relay motor information outward from the spinal cord 402.

It will be understood that the leads, systems, and methods described herein are not dependent on any particular biological theory or theory of operation or effect. Moreover, the leads, systems, or methods should not be understood, interpreted, or viewed in relation to any particular biological theory or theory of operation or effect, unless indicated otherwise.

It is thought that in chronic pain states microglia may assume an activated hypertrophic phenotype, may proliferate, and may contribute to the maintenance of chronic pain. Nerve injury may activate microglia in the dorsal horn of the spinal cord. It has been suggested that the resultant hyperexcitability in the dorsal horn pain network induced by factors from activated microglia may be at least partially responsible for neuropathic pain. Moreover, it is thought possible that activated glial cells, astrocytes and microglia within the spinal cord could maintain the pain sensation even after the original injury or inflammation has healed, and convert it into chronic by altering neuronal excitability.

It is also found that microglia can be highly motile cells that may migrate hundreds of micrometers toward damaged or infected sites as a response to a number of chemical species, such as, for example, ATP/ADP or lysophosphatidic acid. It has been suggested that microglia respond to extracellular ATP by releasing ATP to provide a positive feedback mechanism to generate a long-range extracellular signal for attracting distant microglia to migrate towards and accumulate at the site of an injury.

It has been suggested that pain-related glial cells in the spinal cord are often largely isolated to the dorsal horns. Conventional paddle leads are typically arranged with electrodes for dorsal column stimulation. In contrast, in at least some embodiments, a paddle lead for glial cell stimulation can include electrodes spaced further apart mediolaterally in order to be positioned over or near the dorsal horns. In some embodiments, a paddle lead may include one or multiple columns for one or both of the lateral aspects and, optionally, a medial sensor array or an array of non-electrode stimulators or any combination thereof.

In at least some embodiments, the electrodes may also be longer in the longitudinal direction than electrodes used for neuronal stimulation. It is believed that shorter electrodes are more likely to stimulate neurons which are activated by variations in the electric field (for example, variations indicated by the second difference of the electric field). In contrast, in at least some embodiments, longer electrodes can produce an electrical field effect to facilitate cytotaxis of glial cells, optionally without (or with reduced) neuronal stimulation. The term “electrical field effect” includes, but is not limited to, an electric field or an electrical stimulation field. An electrical field effect may also produce effects other than stimulation including, but not limited to, cytotaxis, electrotaxis, galvanotaxis, chemotaxis, Lorentz forces that may modulate the release of gliomodulators, or the like.

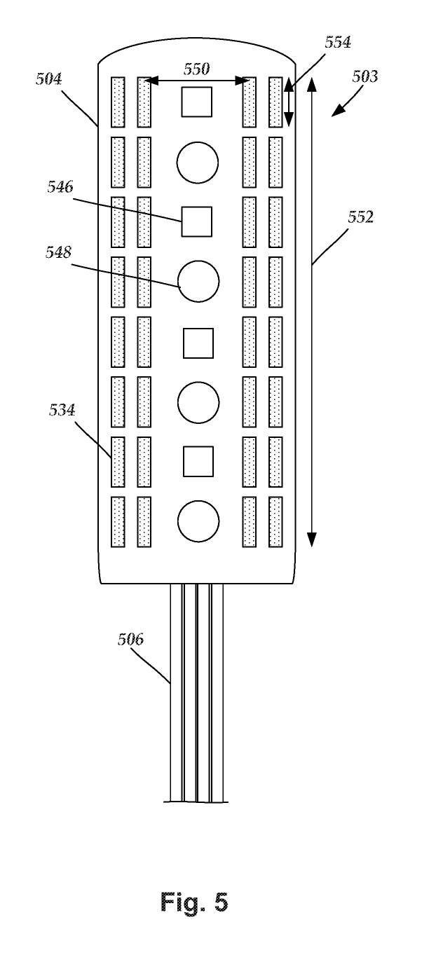

FIG. 5 illustrates one embodiment of a paddle lead 503 with a paddle body 404, multiple columns of electrodes 534, and one or more lead bodies 506 extending from the paddle body 504. The illustrated embodiment also includes one or more optional sensors 546 and one or more optional optical stimulators 548.

The paddles lead 503 includes multiple columns of electrodes 534 along the paddle body 504 with the columns spaced further apart laterally than electrodes used for dorsal column stimulation because the dorsal horns are outside the dorsal column. For example, to stimulate the dorsal horn the columns of electrodes can be spaced apart laterally by a distance 550 of, for example, 7 to 12 mm (center-to-center) or more. This distance may vary depending on the position in the spinal cord where the paddle lead is intended for implantation. Many conventional paddle leads have a total lateral width of no more than 8 mm or less and, therefore, center-to-center lateral spacing between two columns of electrodes for such leads is typically in the range of 6 mm or less.

In the illustrated embodiment, the paddle lead 503 has two columns of electrodes 534 on each lateral side. It will be understood that in other embodiments, there may be one, three, four, or more columns on each side and that the number of columns on each lateral side may be the same or different. In addition, the electrodes 534 in the columns may be aligned with each other, as illustrated in FIG. 5, or may be staggered relative to each other. In at least some embodiments, the columns of electrodes 534 are selected to be relatively long when compared to the electrodes of conventional paddle leads to facilitate cytotaxis of glial cells along a migratory path defined by spatial and temporal variation of the stimulation along the length of the paddle lead. For example, the length 552 of the column of electrodes 534 can be at least 120, 125, 130, 132, 135, 140, 145, or 150 mm or more.

Each of the columns can include any number of electrodes 534 including, but not limited to, one, two, three, four, five, six, seven, eight, nine, ten, twelve, sixteen, or more electrodes. The electrodes 534 in each of the columns can be spaced apart longitudinally in a uniform manner, as illustrated in FIG. 5, or in any other regular or irregular pattern. The electrodes 534 can be identical in size and shape or differ in size or shape. The columns may have the same number of electrodes 534a or different numbers of electrodes. The columns can be identical with respect to arrangement of the electrodes 534a or can be different. In at least some embodiments, one or more (or all) of the electrodes 534 are selected to be relatively long when compared to electrodes of conventional paddle leads to facilitate stimulation of glial cells with reduced or no stimulation of neuronal cells. For example, the length 554 of the electrode 534 can be at least 4, 5, 6, 7, or 8 mm or more. In at least some embodiments, the longitudinal spacing between electrodes in a column is at least 0.5 mm.

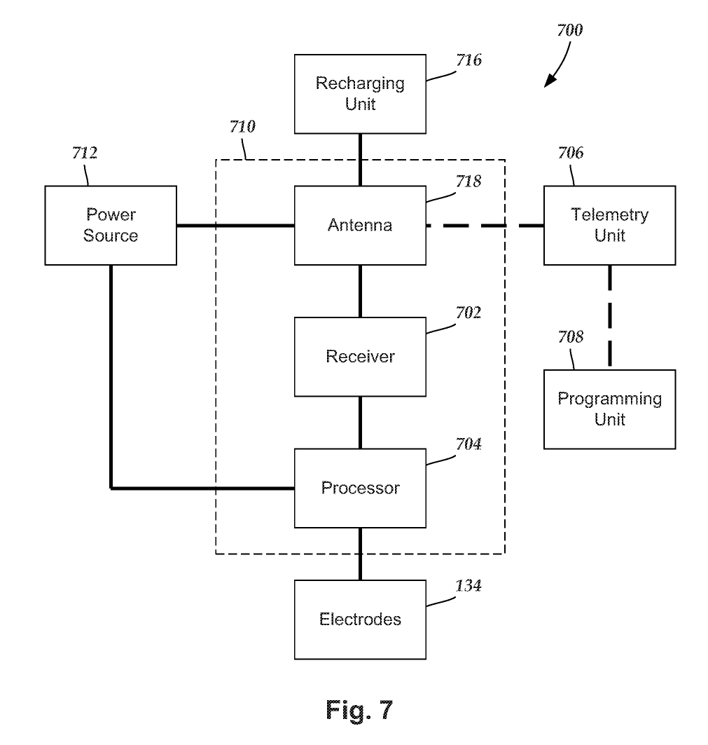

The optional sensors 546 can be any suitable type of sensor including, but not limited to, optical sensors, piezoelectric sensors, chemical sensors, accelerometers, or the like. The paddle lead may include one, two, three, four, or more sensors. The sensors may be identical or may be different (for example, different types of sensors or sensors for different types of chemicals or signals). In at least some embodiments, a sensor 546 is connected to one or more terminals (for example, terminals 310 of FIG. 3A or 3B or additional terminals) at the proximal end of the lead via one or more conductors. Alternatively or additionally, a sensor 546 can be wirelessly coupled to a control module, programming unit (see, FIG. 7), or any other suitable device using Bluetooth™, rf transmission, or any other suitable transmission arrangement.

The optional optical stimulators 548 can be any suitable type of optical stimulator including, but not limited to, light emitting diodes (LEDs), organic light emitting diodes (OLEDs), or the like, or the terminal end of an optical fiber that is coupled, or coupleable, to a light source in the paddle lead or control module or other device. The paddle lead may include one, two, three, four, or more optical stimulators. The optical stimulators may be identical or may be different (for example, emit different wavelengths of light). A paddle lead may include one or more sensors or one or more optical stimulators or any combination thereof. In at least some embodiments, an optical stimulator 548 is connected to one or more terminals (for example, terminals 310 of FIG. 3A or 3B or additional terminals) at the proximal end of the lead via one or more conductors. If the optical stimulator is an optical fiber, the optical fiber may be coupled to an optical terminal at the proximal end of the lead (for example, when the light source is external to the lead such as located in the control module or other device) or to a light source, such as a light emitting diodes (LEDs), organic light emitting diodes (OLEDs), or the like within the lead.

In some embodiments, the paddle lead 503 can include additional medial electrodes (not shown) for stimulation or for sensing. These additional electrodes can be formed as one or more columns and may be the same size as the electrodes 534 or may be sized differently (for example, to stimulate neuronal tissue by being smaller in longitudinal length).

Percutaneous or isodiametric leads can be used instead of, or in addition to, paddle leads. One or more percutaneous leads can be implanted for spinal cord stimulation. For example, one or more percutaneous leads can be implanted on each lateral side of the spinal cord and arranged over or near the dorsal horns. Optionally, a medial lead may also be implanted.

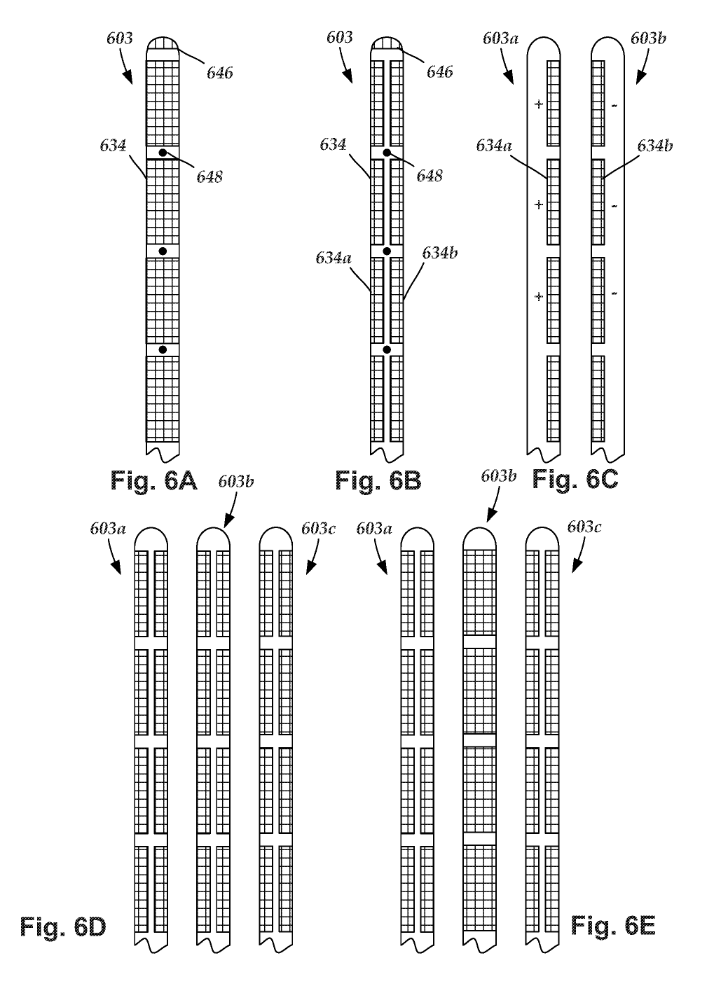

FIG. 6A illustrates one embodiment of a percutaneous lead 603 with multiple electrodes 634, one or more optional sensors 646, and one or more optional optical stimulators 648. In the embodiment of FIG. 6A, the electrodes 634 are cylindrical. In at least some embodiments, the set of electrodes 634 are selected to be relatively long when compared to the electrodes of conventional percutaneous leads to facilitate cytotaxis of glial cells along a migratory path defined by spatial and temporal variation of the stimulation along the length of the paddle lead. For example, the length from the distal-most electrode to the proximal-most electrode can be at least 120, 125, 130, 132, 135, 140, 145, or 150 mm or more.

The lead 603 can include any number of electrodes 634 including, but not limited to, one, two, three, four, five, six, seven, eight, nine, ten, twelve, sixteen, or more electrodes. The electrodes 634 can be spaced apart longitudinally in a uniform manner, as illustrated in FIG. 6, or in any other regular or irregular pattern. The electrodes 634 can be identical in size and shape or differ in size or shape. In at least some embodiments, one or more (or all) of the electrodes 634 are selected to be relatively long when compared to electrodes of conventional percutaneous leads to facilitate stimulation of glial cells with reduced or no stimulation of neuronal cells. For example, the length of the electrode 634 can be at least 4, 5, 6, 7, or 8 mm or more. In at least some embodiments, the longitudinal spacing between electrodes is at least 0.5 mm.

The optional sensors 646 and optional optical stimulators 648 can be any of those described above with respect to the embodiment illustrated in FIG. 5. Moreover, the optional sensors 646 and optional optical stimulators 648 can be positioned at the tip of the lead, between electrodes, or proximal to all of the electrodes, or any combination thereof and are not limited to the positions illustrated in FIG. 6. A percutaneous lead may include one or more sensors or one or more optical stimulators or any combination thereof. In at least some embodiments, a sensor 646 is connected to one or more terminals (for example, terminals 310 of FIG. 3A or 3B or additional terminals) at the proximal end of the lead via one or more conductors. Alternatively or additionally, a sensor 646 can be wirelessly coupled to a control module, programming unit (see, FIG. 7), or any other suitable device using Bluetooth™, rf transmission, or any other suitable transmission arrangement. In at least some embodiments, an optical stimulator 648 is connected to one or more terminals (for example, terminals 310 of FIG. 3A or 3B or additional terminals) at the proximal end of the lead via one or more conductors. If the optical stimulator is an optical fiber, the optical fiber may be coupled to an optical terminal at the proximal end of the lead (for example, when the light source is external to the lead such as located in the control module or other device) or to a light source, such as a light emitting diodes (LEDs), organic light emitting diodes (OLEDs), or the like within the lead.

FIG. 6B illustrates another embodiment of a percutaneous lead 603 with multiple electrodes 634, one or more optional sensors 646, and one or more optional optical stimulators 648. In the embodiment of FIG. 6, the electrodes 634 are segmented electrodes and do not extend around the entire circumference of the lead. In the illustrated embodiment of FIG. 6B, there is a set of two segmented electrodes 634a, 634b at each longitudinal position along the lead. Each of these segmented electrodes 634a, 634b extends no more than 160, 150, 145, 140, 135, 100, or 90 degrees around the circumference of the lead 603. In other embodiments, there may be three, four, or more electrodes in each set. In at least some embodiments, each set of electrodes includes the same number of segmented electrodes. Alternatively, the sets of electrodes may include different numbers of segmented electrodes.

Segmented electrodes may provide for superior current steering than cylindrical or ring electrodes because target structures in electrical stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to 1 target tissue, while potentially avoiding stimulation of other tissue. Examples of leads with segmented electrodes include U.S. Pat. Nos. 8,473,061; 8,571,665; and 8,792,993; and U.S. Patent Application Publications Nos. 2010/0268298; 2011/0130803; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358207; 2014/0358208; 2014/0358209; 2014/0358210; 2015/0045864; 2015/0066120; 2015/0018915; 2015/0051681; 2015/0151113; and 2016/0228692, all of which are incorporated herein by reference in their entireties.

In some embodiments, a lead may include a combination of segmented electrodes (FIG. 6B) and cylindrical or ring electrodes (FIG. 6A). Any suitable combination and arrangement of these types of electrodes can be used. The arrangement, length of the array of electrodes, length of individual electrodes, and longitudinal spacing between the electrodes an embodiment with segmented electrodes can be the same as described above for the embodiment illustrated in FIG. 6A.

FIG. 6C illustrates an arrangement of two percutaneous leads 603a, 603b with segmented electrodes 634a, 634b that are implanted adjacent to each other. For example, the lead 603a can be implanted over the dorsal horn in a lateral position and the lead 603b can be implanted medially with respect to the spinal cord. Alternatively, the two leads 603a, 603b can be implanted laterally with respect to the spinal cord (for example, over or near the two dorsal horns.)

This arrangement can be particularly useful for generating mediolateral bipolar fields by selecting one or more of the electrodes on one lead as anodes and one or more electrodes of the other lead as cathodes. One example of such an arrangement is indicated by the distribution of “+” and “−” signs in FIG. 6C. By using multiple electrodes of each lead, a broad region with an electrical field effect in a medial aspect can be generated to facilitate cytotaxis of glial cells. Of course, a similar arrangement can be made using the leads of either FIG. 6A or 6B (or a combination of these two leads).

FIGS. 6D and 6E illustrate two arrangements of three percutaneous leads 603a, 603b, 603c. For example, the leads 603a, 603c can be implanted laterally with respect to the spinal cord (for example, over or near the two dorsal horns) and the lead 603b can be implanted medially. These arrangements may provide for further control to produce mediolateral fields or produce fields that can be used to migrate or steer glial cells or glial functionality along the regions of the spinal cord covered by the leads.

In at least some embodiments, the lead can be designed for intradural placement. In at least some embodiments, the lead may include a drug eluting lumen or drug-delivering surface, for example, to promote gliomodulatory effects, such as chemotaxis, in response to drug elution or to produce pro- or anti-inflammatory effects or any combination thereof.

Alternatively, or in addition, to stimulation of the dorsal horn region, one or leads (percutaneous or paddle leads) may be provided for stimulation of the dorsal columns, dorsal column nuclei and caudal medulla (with cervical implant), dorsal roots, spinocerebellar (dorsolateral) tracts, for example, for proprioception, dorsal root ganglia (DRG) or satellite ganglion cells, dorsolateral funiculus, peripheral nerves and Schwann cells, ventral horns, ventral roots, ventrolateral tracts (e.g. anterolateral STT, for example, for pain modulation), ventromedial tracts (rubrospinal, medullary pyramidal tract) for example, for motor control and modulation, or any combination thereof. In some embodiments, stimulation for multiple regions may be provided by the same lead. In some embodiments, stimulation of different regions may be provided using different leads.

Electrode configuration and migration of fields can be used to produce gliomodulatory effects. Monopolar, bipolar, tripolar, or other multipolar target poles can be selected to facilitate behaviors by microglia, astrocytes, and/or oligodendrocytes to promote healing, reduce inflammation, or relieve pain, or produce any combination of these effects. The cellular behaviors that can be produced include, but are not limited to, electrotaxis, galvanotaxis, chemotaxis, thermogenic effects, and other electromechanical effects such as those produced by Lorentz interactions caused by an applied waveform.

It is thought that Lorentz forces may modulate the release of gliomodulators. For example, monopolar, bipolar, tripolar, or other multipolar target poles can be configured so that the amplitude and orientation of the target pole can induce a directional Lorentz force on glial cells with any morphology. In at least some embodiments, the system may allow a user to define (or the system may automatically define) a target pole, or a set or series of target poles, to induce morphological changes in glial cells that can result in the release of gliomodulators. For example, a user can define a starting field, a path (or one or more spatial vectors), an ending region or end of the path, and a migration rate or path duration, or any combination thereof. In addition, the user may also define whether the path repeats and the rate of repetition. In some embodiments, the target poles may be fit to the existing electrodes using lead squares or other fitting algorithms or methods. The waveform shape or pulse pattern may also be defined to produce the desired glial effect.

In at least some embodiments, monopolar or multipolar configurations can be used to simultaneously produce neuronal and glial effects that act in a complementary manner, such as, for example, a field orientation to produce synaptic activation and a field orientation to promote selective electrotaxis towards or away from a point in the neural structure.

In at least some embodiments, multi-area stimulation can be used to produce neuronal and glial effects in different regions of the target tissue that may complement each other using the same waveforms (that could be staggered, cycled, or otherwise temporally offset) or different waveforms. For example, spread bipoles in the rostrocaudal direction or strip bipoles in the mediolateral direction or any combination thereof can be effective and may be selected to avoid or provide relatively little neuronal stimulation. In at least some embodiments, migratory fields can be used where distinct sets of contacts along the lead are successively activated over fixed or variable intervals to deliver a specific phase of a given waveform, pulse pattern, of the like to a specific part of the region of interest and migrated in a way to encourage electrotaxis.

In addition to, or as an alternative to, defining spatial elements of the stimulation, temporal elements of the stimulation can also be defined. For example, a pulse frequency (for example, in a range of 2 Hz to 10 kHz), a pulse width (for example, in range of 10 microseconds to 1 millisecond or more), an amplitude (for example, in a range of 0.1 to 25 mA or more), or a pulse shape (for example, square, sawtooth, sinusoidal, or any other suitable shape including user-define or template shapes that can be regular or irregular), or any combination of these features can be user-defined or system-defined.

As an example, long pulse width, low slew rate waveforms over several contacts can be scheduled in order to evoke gradual migration of glial populations to/from regions of the spinal cord. It is thought, for example, that long pulse width, low slew rate waveforms may depolarize the synapse and also induce a desired glial effect such as electro- or chemo-taxis, or glially mediated changes in synaptic strength (for example, via neurotransmitter binding to sites on glia, modulation of calcium dynamics).

The system may allow program schedules to be built for a patient, optionally based on patient-acquired data, or downloaded (or otherwise obtained) from a database. In at least some embodiments, these program schedules may be configurable by the user (e.g., a programmer, clinician, or patient.) The program schedule may cycle between sets of waveforms or patterns or other parameters (or any combination of parameters) over time. This cycling may be pre-determined or may be modified by a user or the system to produce a desired effect. Prolonged gliomodulation may result in depletion of gliotransmitters. Such an effect may be mitigated, and energy use may be reduced by cycling stimulation (for example, cycling based upon known or derived glial depletion curves).

In at least some embodiments, a noise signal may be delivered on top of another waveform (for example a square, sawtooth, or sinusoidal waveform) to encourage stochastic resonance. This may provide a subthreshold enhancement of a desired stimulation effect at a suprathreshold level. For example, a low amplitude waveform could be applied simultaneously with a higher amplitude waveform to produce a subthreshold effect, such as glial electromechanical modulation that enhances or makes supra-threshold an effect due to the presence of the higher amplitude waveform that would otherwise not have occurred. In at least some embodiments, the low- and high-amplitude waveforms may be designed based on a model or machine learning from clinical data for producing complementary neuron-glial effects. In at least some embodiments, random pulses may be interspersed onto a regular waveform to produce subthreshold effects on neurons or glia or any combination thereof that may, upon introduction of a regular pulse, produce or enhance a desired effect.

In at least some embodiments, the system may be arranged to produce multimodal gliomodulation combining two or more of electrical, optical, thermal, acoustic, or chemical stimulation.

FIG. 7 is a schematic overview of one embodiment of components of an electrical stimulation system 700 including an electronic subassembly 710 disposed within a control module. It will be understood that the electrical stimulation system can include more, fewer, or different components and can have a variety of different configurations including those configurations disclosed in the stimulator references cited herein.

Some of the components (for example, a power source 712, an antenna 718, a receiver 702, and a processor 704) of the electrical stimulation system can be positioned on one or more circuit boards or similar carriers within a sealed housing of an implantable pulse generator, if desired. Any power source 712 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 712 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 704 is generally included to control the timing and electrical characteristics of the electrical stimulation system. For example, the processor 704 can, if desired, control one or more of the timing, frequency, strength, duration, and waveform of the pulses. In addition, the processor 704 can select which electrodes can be used to provide stimulation, if desired. In some embodiments, the processor 704 selects which electrode(s) are cathodes and which electrode(s) are anodes. In some embodiments, the processor 704 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 708 that, for example, allows modification of pulse characteristics. In the illustrated embodiment, the processor 704 is coupled to a receiver 702 which, in turn, is coupled to the optional antenna 718. This allows the processor 704 to receive instructions from an external source to, for example, direct the pulse characteristics and the selection of electrodes, if desired.

In one embodiment, the antenna 718 is capable of receiving signals (e.g., RF signals) from an external telemetry unit 706 which is programmed by the programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the electrical stimulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and the receiver 702 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 700 to cease operation, to start operation, to start charging the battery, or to stop charging the battery. In other embodiments, the stimulation system does not include the antenna 718 or receiver 702 and the processor 704 operates as programmed.

Optionally, the electrical stimulation system 700 may include a transmitter (not shown) coupled to the processor 704 and the antenna 718 for transmitting signals back to the telemetry unit 706 or another unit capable of receiving the signals. For example, the electrical stimulation system 700 may transmit signals indicating whether the electrical stimulation system 700 is operating properly or not or indicating when the battery needs to be charged or the level of charge remaining in the battery. The processor 704 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A paddle lead, comprising:
a paddle body;
a first column comprising a plurality of electrodes disposed along the paddle body;
a second column comprising a plurality of electrodes disposed along the paddle body;
at least one of a non-electrical sensor or an optical stimulator disposed along the paddle body and between the first and second columns;
at least one lead body extending from the paddle body; and
a plurality of terminals disposed along the at least one lead body and electrically coupled to the plurality of electrodes of the first and second columns.

2. The paddle lead of claim 1, wherein the at least one of the non-electrical sensor or the optical stimulator comprises a plurality of the non-electrical sensors arranged in a medial column between the first and second columns.

3. The paddle lead of claim 1, wherein the at least one of the non-electrical sensor or the optical stimulator comprises a plurality of the optical stimulators arranged in a medial column between the first and second columns.

4. The paddle lead of claim 1, wherein the at least one of the non-electrical sensor or the optical stimulator comprises a plurality of the non-electrical sensors and the optical stimulators arranged in a medial column between the first and second columns.

5. The paddle lead of claim 1, wherein the at least one of the non-electrical sensor or the optical stimulator comprises at least one of the non-electrical sensors selected from an optical sensor, a piezoelectric sensor, a chemical sensor, or an accelerometer.

6. The paddle lead of claim 1, wherein the at least one non-electrical sensor or optical stimulator comprises at least one of the optical stimulators selected from a light emitting diode, an organic light emitting diode, or an optical fiber coupleable to a light source.

7. The paddle lead of claim 1, wherein at least one of the electrodes has a dimension of at least 4 mm.

8. The paddle lead of claim 1, wherein the first column of electrodes comprises no more than eight of the electrodes and the first column has a dimension of at least 120 mm from a distal end of a distal-most one of the electrodes to a proximal end of the proximal-most one of the electrodes.

9. The paddle lead of claim 1, wherein the first and second columns are separated by a center-to-center distance of at least 7 mm.

10. The paddle lead of claim 1, wherein the first and second columns are configured and arranged to modulate glial cells preferentially over neurons.

11. An electrical stimulating system comprising:
the paddle lead of claim 1; and
a control module coupleable to the paddle lead, the control module comprising
a housing, and
an electronic subassembly disposed in the housing.

12. A percutaneous lead, comprising:
a lead body having a distal portion and a proximal portion;
a plurality of electrodes disposed along the distal portion of the lead body;
at least one non-electrical sensor disposed along the distal portion of the lead body;
at least one optical stimulator disposed along the distal portion of the lead body; and
a plurality of terminals disposed along the proximal portion of the lead body and electrically coupled to the plurality of electrodes.

13. The percutaneous lead of claim 12, wherein the at least one non-electrical sensor is selected from an optical sensor, a piezoelectric sensor, a chemical sensor, or an accelerometer.

14. The percutaneous lead of claim 12, wherein the at least one optical stimulator comprises at least one of a light emitting diode, an organic light emitting diode, or an optical fiber coupleable to a light source.

15. The percutaneous lead of claim 12, wherein at least one of the electrodes has a dimension of at least 4 mm.

16. The percutaneous lead of claim 12, wherein the plurality of electrodes comprises no more than eight of the electrodes with a dimension of at least 120 mm from a distal end of a distal-most one of the electrodes to a proximal end of the proximal-most one of the electrodes.

17. The percutaneous lead of claim 12, wherein the electrodes are configured and arranged to modulate glial cells preferentially over neurons.

18. An electrical stimulating system comprising:
the percutaneous lead of claim 12; and
a control module coupleable to the percutaneous lead, the control module comprising
a housing, and
an electronic subassembly disposed in the housing.

19. A method for glial modulation, the method comprising:
implanting the paddle lead of claim 1 adjacent a spinal cord of a patient; and
delivering electrical stimulation through one or more of the electrodes of the paddle lead to modulate glial cells in the spinal cord.

20. A method for glial modulation, the method comprising:
implanting the percutaneous lead of claim 12 adjacent a spinal cord of a patient; and
delivering electrical stimulation through one or more of the electrodes of the percutaneous lead to modulate glial cells in the spinal cord.
